(19) United States
(12) Patent Application Publication
(10) Pub. No.: US 2024/0387035 A1
(43) Pub. Date: Nov. 21, 2024

(54) Compliance Methodology And System To Verify Caregiver Service Delivery For Chronic Care Management

(71) Applicant: Javier Vinals, Irving, TX (US)

(72) Inventor: Javier Vinals, Irving, TX (US)

(21) Appl. No.: 18/199,378

(22) Filed: May 19, 2023

(51) Int. Cl. G16H 40/67; G06F 21/31; G06F 21/44; G16H 10/60

(52) U.S. Cl. CPC G16H 40/67; G06F 21/31; G06F 21/44; G16H 10/60; G06F 2221/2151

(57) ABSTRACT

This invention relates in general to a physician directed patient care and measurement reporting Electronic Health Records (EHR) systems and supporting applications which enable structured online caregiver to patient interaction. The proposed methodology and system provides secure access and authentication of caregiver access and service engagement to assigned patients for the purpose of providing care plan directed activities that meet the compliance standards set forth by regulatory agencies.

### References (U.S. Patent):

| 7,552,467 | June 2009 Lindsay May 2010 Ballard July 2014 | |
|---|---|---|
| 7,725,732 | Khurana et al. | |
| 8,793,790 | February 2011 | Allen et al. |
| 2011/0047377 | March 2012 | Mardikar et al. |
| 2012/0060207 | August 2012 | Hannel et al. |
| 2012/0198232 | September 2012 | Davis et al. |
| 2012/0239950 | February 2014 | Nitz et al. |
| 2014/0052681 | July 2016 | Schultz, et al. |
| 2016/9391986 | | |

Other References:
a. “Chronic Care Management Services, Changes 2017”, Centers for Medicare and Medicaid Services (cms.gov)
b. “A Strong Pulse for Biometrics in Healthcare”, (PlanetBioMetrics.Com, 2013September 27)
c. “Biometric Identity in Healthcare: Reduces Health Care Fraud, Improves Patient Care and Protects Patient Privacy”, (IBIA, 2011 July).
d. “Healthcare Biometric Identity Management Technology”, Versos, Sep. 20, 2013
e. “Positive Outcomes Implementing Biometrics in Multiple Healthcare”, Schneider, John K. (2011).
f. “Smart Cards and Biometrics in Healthcare Identity Applications”, (Smart Card Alliance, 2012 May).
g. “Knowledge Management in Health Care: an Architectural Framework for Clinical Process Management Systems”, DEXA, IEEE Computer Society, 2005

### REFERENCE TO RELATED PATENT APPLICATIONS

This application claims the benefit of U.S. Provisional Pat. App. Ser. No. 62/528,852, filed on Jul. 5, 2017, entitled, “Interactive Template Driven System to Support Physician Directed Chronic Care Plan Management” of which certain core functions are referenced herein as an “online caregiver and patient engagement network” functioning as an overall patient relationship management system.

### DESCRIPTION

### FIELD OF THE INVENTION

This invention relates in general to a physician directed patient care and measurement reporting Electronic Health Records (EHR) systems and supporting applications which enable structured online caregiver to patient interaction. The proposed methodology and system provides secure access and authentication of caregiver access and service engagement to assigned patients for the purpose of providing care plan directed activities that meet the compliance standards set forth by regulatory agencies. This system feeds and extracts data from a plurality of patient information exchange and storage sub-systems and applications including EMR, EHR and PHR systems.

### BACKGROUND OF THE INVENTION

Chronic Care Management (CCM) is defined as the non-face-to-face services provided to Medicare beneficiaries who have two or more significant chronic conditions. In addition to office visits and other face-to face encounters, which must be billed separately, these services include communication with the patient and other treating health professionals for care coordination (both electronically and by phone), medication management, and providing accessibility 24 hours a day to patients and other treating physicians or clinical staff. The creation and revision of electronic care plans is also a key component of CCM.

Based on government requirements and mandates, the designated CCM clinician (MD, PA, NP) must establish, implement, revise, or monitor and manage an electronic care plan that addresses the physical, mental, cognitive, psychosocial, functional, and environmental needs of the patient as well as maintain an inventory of resources and supports that the patient needs. The work involves motivating patients to persist in necessary therapies and interventions and helping them to achieve an ongoing, reasonable quality of life.

Historically, reimbursement has been challenging for chronic care coordination services. Medicare recently started paying for services related to chronic care management. Medicare pays a monthly fee for patients who consent to treatment for a minimum of 20 minutes of telehealth services. The present invention proposes a methodology which can be incorporated into the process flows for a plurality of healthcare systems which provide a functional caregiver to patient service delivery verification and assurance means to provide documented (auditable) evidence of compliance with government regulated care delivery mandates and guidelines for patient chronic care treatment.

### DESCRIPTION

### FIELD OF THE INVENTION

The present invention relates in general to online patient chronic care plan management and service delivery, and in particular, to a methodology applicable to a plurality of systems and applications to ensure the monitoring, verification and record validation for caregiver to patient engagement supporting care plan directed activities.

### BRIEF DESCRIPTION OF THE DRAWINGS

FIG. 1: is a system block diagram of a typical physician to patient electronic health record access system and associated EMR subsystems.

FIG. 2: illustrates the service assurance validation methodology and flow for the system.

### DETAILED DESCRIPTION OF THE PREFERRED EMBODIMENT

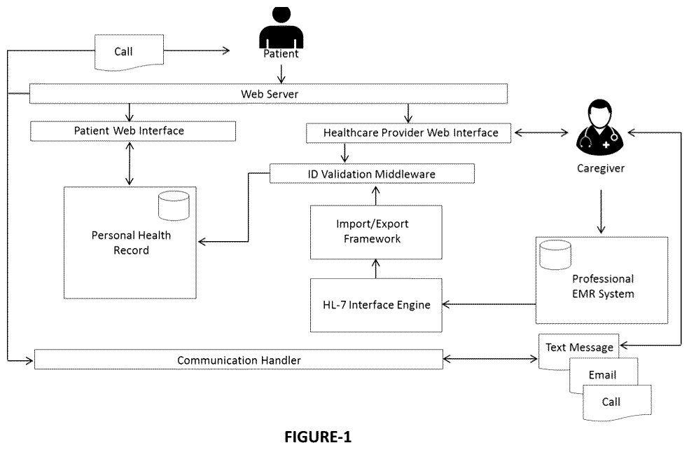

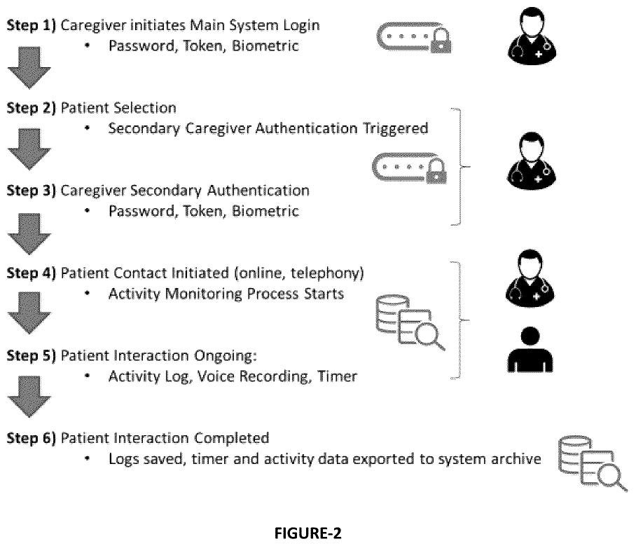

An online caregiver and patient engagement network system (FIG. 1) that is configured to: 1) Provide active verification of authorized caregiver access. 2) Enable caregiver to select a specific CCM patient for interaction. 3) Initiate secondary caregiver authentication (FIG. 2) to engage the selected patient in online or telephony based communication. 4) A timed validation record (FIG. 2) is initiated and maintained during the service delivery of the care plan activity with the patient that is stored for verification and archival reporting to the main caregiver system.

What is claimed is:

1. A service assurance methodology and interactive system to verify and validate that Chronic Care Management (CCM) services are delivered according to government directed time allocation mandates using a plurality of access control devices and monitoring methods including biometrics, electronic time stamping, e-badge, password and other communications and application access authentication technologies.

2. The interactive chronic care plan engagement system of claim 1 wherein the assigned caregiver must authenticate their permission level and login to the system to access chronic care patient application data and engagement platform.

3. The interactive chronic care plan engagement system and application of claim 1 wherein the caregiver can select an individual patient in the Chronic Care system for direct Care Plan interaction.

4. The interactive chronic care plan engagement system and application of claim 1, wherein the caregiver undergoes secondary authentication to initiate patient contact and execute assigned Care Plan activities with the patient using a plurality of authentication methods including:
Password.
Smart-card
Single Use System Issued Token
Biometric Identification including: fingerprint, face, retinal, and iris recognition

5. The interactive chronic care plan engagement application system of claim 1, wherein upon patient selection, caregiver authentication and active patient engagement, monitoring is initiated and a timed record of the care plan services interaction is executed for the caregiver and patient interaction which may include audio/video and keystroke recording and a timestamp for event duration.

6. The interactive chronic care plan engagement system of claim 1, wherein a caregiver authentication, activity and time stamp record of the care plan engagement activity with the patient is recorded and output to an online EMR/EHR system for storage.
